(19) United States
(12) Patent Application Publication
(10) Pub. No.: US 2011/0288196 A1
(43) Pub. Date: Nov. 24, 2011

(54) DENTAL COMPOSITIONS COMPRISING BISACRYLAMIDES AND USE THEREOFE

(76) Inventors: Uwe Walz, Konstanz (DE); Joachim E. Klee, Radolfzell (DE)

(21) Appl. No.: 13/196,289

(22) Filed: Aug. 2, 2011

Related U.S. Application Data
(63) Continuation of application No. 12/586,789, filed on Sep. 28, 2009, now abandoned.
Application No. 12/586,789 is a continuation of application No. 11/699,597, filed on Jan. 29, 2007, now abandoned.
Application No. 11/699,597 is a continuation-in-part of application No. 10/308,564, filed on Dec. 3, 2002, now Pat. No. 6,767,936.

(51) Int. Cl. A61K 6/083; C08F 2/46; C08L 35/00

(52) U.S. Cl. 522/48; 523/118; 522/175; 523/116

(57) ABSTRACT

The invention concerns a dental composition comprising bisacrylamides that are polymerizable by free-radical polymerization and by Michael-addition polymerization with amines. The application of polymerization and addition polymerization with amines opens the possibility to completely reacted materials that have no oxygen inhibited layers. The claimed bisacrylamides have an improved hydrolytic stability.

### RELATED APPLICATIONS

This application is a continuation of application Ser. No. 12/586,789 filed Sep. 28, 2009, which is a continuation of Ser. No. 11/699,597 filed Jan. 29, 2007 which is a continuation-in-part of divisional application Ser. No. 10/308,564 filed Dec. 3, 2002 (Case KON-77A DIV) which claims the benefit of Ser. No. 09/925,173 filed Aug. 9, 2001 (Case KON-77A) which claims the benefit of U.S. Provisional Application Ser. No. 60/310,572 filed Aug. 7, 2001 (Case KON-77A PRO) which claims the benefit of U.S. Provisional Application Ser. No. 60/224,670 filed Aug. 11, 2000 (Case KON-77A PRO).

### TECHNICAL BACKGROUND

Since decades the free-radical polymerization used in electrotechnics, electronics, dental industry, is combined with remarkable advantages in these fields. The frequently used acrylates and methacrylates are applied in combination with pigments and fillers or as pure polymerizable resins. It is well-known that during free-radical polymerization some side-reactions take place. One of them is the inhibition of the outer layer of the polymerizable material due to the influence of oxygen. The thickness of this layer depends on the viscosity of the polymerizable material, the degree of filling, the applied temperature and the time of polymerization. Frequently, the oxygen inhibited layer is disadvantageous due to the mechanical properties in this part are insufficient, the abrasion is higher and the toxicological/allergic potential is increased. The polymerization of very small layers is limited due to the oxygen inhibition, for example in case of covering electronic circuits by screen printing or for dental sealing materials or varnishes.

Furthermore, the conventional methacrylates that were used for dental applications are ester compound. Consequently, they hydrolysis under acidic or basic conditions that frequently leads to a long-term failure.

In order to reduce the oxygen inhibited layer different possibilities were suggested. One of them is the today well-known use of carbonyl/amine initiator systems for photochemical polymerization (R. S. Davison, J. W. Goodin, Eur. Polym. J 18 (1982) 597). Dekker used special color initiators that change triplet-oxygen into singulet-oxygen (C. Dekker, Makromol. Chem. 180 (1979) 2027). Furthermore, surface active additives were used (C. R. Morgan, A. D. Ketley, J. Radiat. Curing 7 (1980) 10) or the photochemical SH-En-Addition was applied (C. R. Morgan, F. Magnotta, A. D. Ketley, J. Polym. Sci., Polym. Ed. 15 (1977), 627).

The photochemical polymerization of monoacrylamides was studied by Smets (G. Smets, Bull. Soc. Chim. Belges 71 (1962) 857, G. Oster, J. Amer. Chem. Soc. 79 (1957) 595). A large number of bisacrylamides were described by Ferrutti (P. Ferrutti et al., Polymer 26 (1985) 1336). These bisacrylamides are solids that are soluble in water due to the secondary amide group or they comprises a piperidine group.

A combination of free-radical and Michael addition polymerization was suggest for encapsulation of electronic circuits (DD 295645; invs.: J. Klee, H.-H. Hörhold, I. Scherlitz-Hofmann).

The new synthesized bisacrylamides should be liquids in order to polymerized them without of solvents and furthermore they and the resulting polymers should be insoluble in water.

### DESCRIPTION OF THE INVENTION

A dental composition that comprises at least one acrylamide selected from bisacrylamide, polyacrylamide, bis(meth)acrylamide and poly(meth)acrylamide; a polymerizable monomer; at least one amine and/or an initiator; a stabilizer; pigments and an organic and/or inorganic filler and that have an improved hydrolysis stability.

The bisacrylamide are characterized by the following formula:

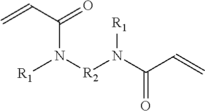

wherein
R₁ is H or a substituted or unsubstituted C₁ to C₁₈ alkylene, substituted or unsubstituted cycloalkylene, substituted or unsubstituted C₅ to C₁₈ arylene or heteroarylene, substituted or unsubstituted C₅ to C₁₈ alkylarylene or alkylheteroarylene, substituted or unsubstituted C₇ to C₃₀ alkylene arylene,
R₂ is a difunctional substituted or unsubstituted C₁ to C₁₈ alkylene, difunctional substituted or unsubstituted cycloalkylene, difunctional substituted or unsubstituted C₅ to C₁₈ arylene or heteroarylene, difunctional substituted or unsubstituted C₅ to C₁₈ alkylarylene or alkylheteroarylene, difunctional substituted or unsubstituted C₇ to C₃₀ alkylene arylene,

Preferably bisacrylamides are characterized by the following formula:

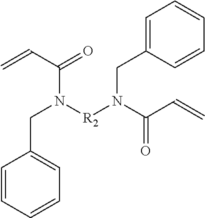

wherein
R₂ is a difunctional substituted or unsubstituted C₁ to C₁₈ alkylene, difunctional substituted or unsubstituted cycloalkylene, difunctional substituted or unsubstituted C₅ to C₁₈ arylene or heteroarylene, difunctional substituted or unsubstituted C₅ to C₁₈ alkylarylene or alkylheteroarylene, difunctional substituted or unsubstituted C₇ to C₃₀ alkylene arylene,

The claimed dental composition preferably contains as polymerizable monomer a mono- or a polyfunctional (meth)-acrylate, such as a polyalkyleneoxide di- and poly-(meth)acrylate, an urethane di- and poly(meth) acrylate, a vinyl-, vinylen- or vinyliden-, acrylate- or methacrylate; preferably were used diethyleneglycol dimethacrylate, triethyleneglycol dimethacrylate, 3, (4),8, (9)-dimethacryloyloxymethyltricyclodecane, dioxolan bismethacry-late, glycerol trimethacrylate, furfuryl methacrylate or a monoacrylamide in a content of 5 to 80 wt-%.

Bisacrylamides react with amines in a thermal Michael addition polymerization. Preferably for the addition polymerization are used primary monoamines, disecondary diamines and/or polyamines of the following structure:

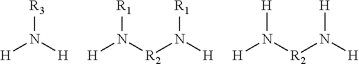

wherein
R₁ is a substituted or unsubstituted C₁ to C₁₈ alkylene, substituted or unsubstituted cycloalkylene, substituted or unsubstituted C₅ to C₁₈ arylene or heteroarylene, substituted or unsubstituted C₅ to C₁₈ alkylarylene or alkylheteroarylene, substituted or unsubstituted C₇ to C₃₀ alkylene arylene,
R₂ is a difunctional substituted or unsubstituted C₁ to C₁₈ alkylene, difunctional substituted or unsubstituted cycloalkylene, difunctional substituted or unsubstituted C₅ to C₁₈ arylene or heteroarylene, difunctional substituted or unsubstituted C₅ to C₁₈ alkylarylene or alkylheteroarylene, difunctional substituted or unsubstituted C₇ to C₃₀ alkylene arylene and
R₃ is a substituted or unsubstituted C₂ to C₁₈ alkylene, substituted or unsubstituted cycloalkylene, substituted or unsubstituted C₅ to C₁₈ arylene or heteroarylene, substituted or unsubstituted C₅ to C₁₈ alkylarylene or alkylheteroarylene, substituted or unsubstituted C₇ to C₃₀ alkylene arylene,

Furthermore the claimed dental composition can contain a polymerization initiator, that preferably is a thermal initiator, a redox-initiator or a photo initiator such as champhor quinone.

In order to avoid a spontaneous polymerization stabilizer are added such as a radical absorbing monomer for example hydrochinon monomethylether (which in English is hydroquinone monoethylether), hydrochinon dimethylether (which in English is hydroquinone dimethylether), 2,6-di-tert.-butyl-p-cresol.

The dental composition comprises an inorganic filler and/or an organic filler. Preferably inorganic fillers such as La₂O₃, ZrO₂, BiPO₄, CaWO₄, BaWO₄, SrF₂, Bi₂O₃, glasses or an organic fillers, such as polymer granulate or a combination of organic/or inorganic fillers are applied.

The dental composition is preferably usable as dental root canal filling material or as pulp capping material.

In an alternative embodiment, the bisacrylamide can have the following formula

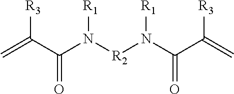

or it can be a polyacrylamide as follows

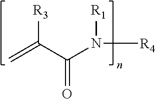

Similarly, the bis(meth)acrylamide can have the following formula

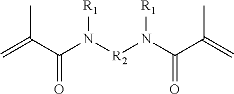

or it can be a poly (meth)acrylamide as follows

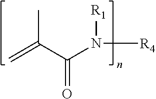

In these formulas, R1 and R3 are the same or different, and are preferably independently H or a substituted or unsubstituted C1 to C18 alkylene, substituted or unsubstituted cycloalkylene, substituted or unsubstituted C5 to C18 arylene or heteroarylene, substituted or unsubstituted C5 to C18 alkylarylene or alkylheteroarylene, substituted or unsubstituted C7 to C30 alkylene arylene; R2 is preferably a difunctional substituted or unsubstituted C1 to C18 alkylene, difunctional substituted or unsubstituted cycloalkylene, difunctional substituted or unsubstituted C5 to C18 arylene or heteroarylene, difunctional substituted or unsubstituted C5 to C18 alkylarylene or alkylheteroarylene, difunctional substituted or unsubstituted C7 to C30 alkylene arylene; and R4 is preferably a mono- or polyfunctional substituted or unsubstituted C1 to C18 alkylene, mono- or polyfunctional substituted or unsubstituted cycloalkylene, mono- or polyfunctional substituted or unsubstituted C5 to C18 arylene or heteroarylene, mono- or polyfunctional substituted or unsubstituted C5 to C18 alkylarylene or alkylheteroarylene, mono- or polyfunctional substituted or unsubstituted C7 to C30 alkylene arylene.

### Example 1

N,N′-bisacryloyl-N,N′-dibenzyl-5-oxanonanediamine-1.9: In a 4-necked 1-l-flask equipped with a stirrer, a thermometer and two 50 ml dropping funnels 102.16 g (0.3 mol) of N,N′-dibenzyl-5-oxanonanediamine-1.9 were dissolved in 300 ml of methylenechloride. After cooling to 0-5° C. 57.020 g (0.63 mol) of acryloylchloride dissolved in 30 ml of methylenechloride and 25.20 g (0.63 mol) of NaOH dissolved in 60 ml of water were added simultaneously under stirring during 1.5 hours so that the temperature remains at 0-5° C. Thereafter the mixture were stirred at room temperature for additional two hours. Than the reaction mixture were hydrolyzed with 600 ml of ice-water. The organic phase were separated and the aqueous solution were extracted twice with methylenechloride. The collected organic liquids were washed with 150 ml of 1 n HCl, 150 ml of 1 n NaHCO₃ and sometimes with 150 ml of deionised water until the water shows a pH-value of approximately 7. Than the organic solution was dried over NaSO₄. Thereafter the NaSO₄ was filtered off and to the solution 0.1346 g of 2,6-di-tert.-butyl-p-cresol were added. The methylenechloride was removed at 40° C. in vacuum and the bisacrylamide was dried.

Yield: 132.6 g (98.5% of th.), n_{D}²⁰=1.5499, η=2.35 Pa*s, Mₙ (vpo)=450 g/mol

C₂₈H₃₆N₂O₃, 448.61 calc C, 74.97; H, 8.09; N, 6.24 found C, 74.50; H, 8.09; N, 6.24

IR: 1655 cm⁻¹ (CONR), 1620 cm⁻¹ (CH₂═CH—)

¹H-NMR: 7.4-7.2 (Ph), 6.65/4.52 (CH₂Ph), 5.58/6.38 (CH₂═CH), 3.4-3.2 (CH₂O, CH₂N), 1.6-1.5 (CH₂CH₂)

¹³C-NMR: 166.69/166.28 (3), 137.60/136.95 (5), 129.66/128.95 (2), 128.80/128.50 (6), 128.35/128.23 (7), 128.16/128.00 (8), 127.27/126.25 (1), 70.40/70.27 (12), 50.99/48.88 (4), 48.07/46.97 (9), 27.43/27.11 (11), 25.43/23.15 (10)

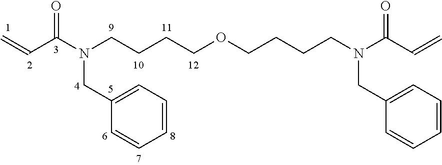

### Addition Polymerization:

5.000 g (11.137 mmol) of N,N′-bisacryloyl-N,N′-dibenzyl-5-oxanonanediamine-1.9 and 3.792 g (11.137 mmol) were mixed homogeneously together and reacted for 60 hours at 60° C. The addition polymer shows the following results obtained by GPC:

| Mₙ | M_{w} | M_{z} | | [η] |
|---|---|---|---|---|
| g mol⁻¹ | g mol⁻¹ | g mol⁻¹ | M_{w}/Mₙ | ml g⁻¹ |
| 3615 | 9403 | 16280 | 2.60 | 8.741 |

### Example 2

N,N′-bisacryloyl-N,N′-dibenzylethylenediamine: In a 4-necked 1-l-flask equipped with a stirrer, a thermometer and two 50 ml dropping funnels 29.198 g (0.12 mol) of N,N′-dibenzylethylenediamine were dissolved in 100 ml of methylenechloride. After cooling to 0-5° C. 21.991 g (0.24 mol) of acryloylchloride dissolved in 30 ml of methylenechloride and 9.718 g (0.24 mol) of NaOH dissolved in 40 ml of water were added simultaneously under stirring during 1.5 hours so that the temperature remains at 0-5° C. Thereafter the mixture were stirred at room temperature for additional two hours. Than the reaction mixture were hydrolyzed with 600 ml of ice-water. The organic phase were separated and the aqueous solution were extracted twice with methylenechloride. The collected organic liquids were washed with 100 ml of 1 n HCl, 100 ml of 1 n NaHCO₃ and sometimes with 100 ml of deionised water until the water shows a pH-value of approximately 7. Than the organic solution was dried over NaSO₄. Thereafter the NaSO₄ was filtered off and to the solution 0.028 g of 2,6-di-tert.-butyl-p-cresol were added. The methylenechloride was removed at 40° C. in vacuum and the bisacrylamide was dried.

Yield: 27.9 g (65.9% of th.), mₚ=75.5-76.6° C., Tg=−7.2° C., (vpo)=350 g/mol

C₂₂H₂₄N₂O₂, 348.45 calc. C, 75.83; H, 6.94; N, 8.04 found C, 76.00; H, 7.26; N, 8.05

### Example 3

N,N′-bisacryloyl-N,N′-dibenzyl-4,4″-diaminodicyclohexylamine: In a 4-necked 1-l-flask equipped with a stirrer, a thermometer and two 50 ml dropping funnels 60.551 g (0.16 mol) of N,N′-dibenzyl-4,4″-diaminodicyclohexylamine were dissolved in 150 ml of methylenechloride. After cooling to 0-5° C. 28.061 g (0.31 mol) of acryloylchloride dissolved in 30 ml of methylenechloride and 12.401 g (0.31 mol) of NaOH dissolved in 50 ml of water were added simultaneously under stirring during 1.5 hours so that the temperature remains at 0-5° C. Thereafter the mixture were stirred at room temperature for additional two hours. Than the reaction mixture were hydrolyzed with 500 ml of ice-water. The organic phase were separated and the aqueous solution were extracted twice with methylenechloride. The collected organic liquids were washed with 100 ml of 1 n HCl, 100 ml of 1 n NaHCO₃ and sometimes with 10 ml of deionised water until the water shows a pH-value of approximately 7. Than the organic solution was dried over NaSO₄. Thereafter the NaSO₄ was filtered off and to the solution 0.077 g of 2,6-di-tert.-butyl-p-cresol were added. The methylenechloride was removed at 40° C. in vacuum and the bisacrylamide was dried.

Yield: 54.0 g (69.9% of th.), Tg=47.1° C.

### APPLICATION EXAMPLE 1

### Dental root canal sealer

### Bisacrylamide-Paste

5.0000 g of N,N′-bisacryloyl-N,N′-dibenzyl-5-oxanonanediamine-1.9 of example 1, 3.1642 g of Calciumtungstate, 0.7911 g of Zirconiumoxide, 0.0300 g of Aerosil and 0.0100 g of Fe₂O₃ were mixed homogeneously.

### Amine-Paste

1.8962 g of N,N′-dibenzyl-5-oxanonanediamine-1.9, 0.8423 g of 1-Aminoadamantane, 10.9540 g of Calciumtungstate, 2.7385 g of Zirconiumoxide and 0.3353 g of Aerosil were mixed homogeneously.

Immediately before use both pastes were mixed homogeneously in a ratio of 1/1 (v/v) or 1/1.86 (w/w). The material shows an radio-opacity of 11.5 mm/mm Al.

### FOREIGN PATENTS

DD 295645; invs.: J. Klee, H.-H. Hörhold, I. Scherlitz-Hofmann

### LITERATURE

R. S. Davison, J. W. Goodin, Eur. Polym. J 18 (1982) 597
C. Dekker, Makromol. Chem. 180 (1979) 2027
C. R. Morgan, A. D. Ketley, J. Radiat. Curing 7 (1980) 10
C. R. Morgan, F. Magnotta, A. D. Ketley, J. Polym. Sci., Polym. Ed. 15 (1977), 627).
G. Smets, Bull. Soc. Chim. Belges 71 (1962) 857
G. Oster, J. Amer. Chem. Soc. 79 (1957) 595
P. Ferrutti et al., Polymer 26 (1985) 1336

We claim:

1. A dental composition that comprises at least a bisacrylamide, a polymerizable monomer, at least an amine and/or an initiator, a stabilizer, pigments and an organic and/or inorganic filler and that have an improved hydrolysis stability.

2. Dental composition of claim 1, wherein said bisacrylamide are characterized by the following formula: 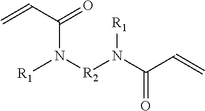
wherein
R₁ is H or a substituted or unsubstituted C₁ to C₁₈ alkylene, substituted or unsubstituted cycloalkylene, substituted or unsubstituted C₅ to C₁₈ arylene or heteroarylene, substituted or unsubstituted C₅ to C₁₈ alkylarylene or alkylheteroarylene, substituted or unsubstituted C₇ to C₃₀ alkylene arylene,
R₂ is a difunctional substituted or unsubstituted C₁ to C₁₈ alkylene, difunctional substituted or unsubstituted cycloalkylene, difunctional substituted or unsubstituted C₅ to C₁₈ arylene or heteroarylene, difunctional substituted or unsubstituted C₅ to C₁₈ alkylarylene or alkylheteroarylene, difunctional substituted or unsubstituted C₇ to C₃₀ alkylene arylene,

3. Dental composition of claim 1, wherein said bisacrylamide are characterized by the following formula: 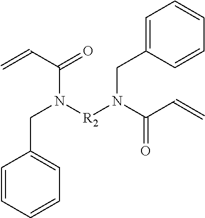
wherein
R₂ is a difunctional substituted or unsubstituted C₁ to C₁₈ alkylene, difunctional substituted or unsubstituted cycloalkylene, difunctional substituted or unsubstituted C₅ to C₁₈ arylene or heteroarylene, difunctional substituted or unsubstituted C₅ to C₁₈ alkylarylene or alkylheteroarylene, difunctional substituted or unsubstituted C₇ to C₃₀ alkylene arylene,

4. Dental composition of claim 1, wherein said polymerizable monomer is a mono- or a polyfunctional (meth)-acrylate, such as a polyalkylenoxide di- and poly-(meth)acrylate, an urethane di- and poly(meth)acrylate, a vinyl-, vinylen- or vinyliden-, acrylate- or methacrylate; preferably were used diethyleneglycol dimethacrylate, triethyleneglycol dimethacrylate, 3,(4),8,(9)-dimethacryloyloxymethyltricyclodecane, dioxolan bismethacry-late, glycerol trimethacrylate, furfuryl methacrylate or a monoacrylamide in a content of 5 to 80 wt-%.

5. Dental composition of claim 1, wherein said wherein said amines are primary monoamines, disecondary diamines and/or polyamines of the following structure: 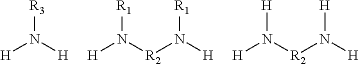
wherein
R₁ is a substituted or unsubstituted C₁ to C₁₈ alkylene, substituted or unsubstituted cycloalkylene, substituted or unsubstituted C₅ to C₁₈ arylene or heteroarylene, substituted or unsubstituted C₅ to C₁₈ alkylarylene or alkylheteroarylene, substituted or unsubstituted C₇ to C₃₀ alkylene arylene,
R₂ is a difunctional substituted or unsubstituted C₁ to C₁₈ alkylene, difunctional substituted or unsubstituted cycloalkylene, difunctional substituted or unsubstituted C₅ to C₁₈ arylene or heteroarylene, difunctional substituted or unsubstituted C₅ to C₁₈ alkylarylene or alkylheteroarylene, difunctional substituted or unsubstituted C₇ to C₃₀ alkylene arylene and
R₃ is a substituted or unsubstituted C₂ to C₁₈ alkylene, substituted or unsubstituted cycloalkylene, substituted or unsubstituted C₅ to C₁₈ arylene or heteroarylene, substituted or unsubstituted C₅ to C₁₈ alkylarylene or alkylheteroarylene, substituted or unsubstituted C₇ to C₃₀ alkylene arylene,

6. Dental composition of claim 1, wherein said polymerization initiator is a thermal initiator, a redox-initiator or a photo initiator.

7. Dental composition of claim 1, wherein said photo initiator preferably is champhor quinone.

8. Dental composition of claim 1, wherein said filler is an inorganic filler and/or an organic filler.

9. Dental composition of claim 1, wherein said stabilizer is a radical absorbing monomer such as hydroquinone monomethylether, hydroquinone dimethylether, 2,6-di-tert.-butyl-p-cresol.

10. Dental composition of claim 1, that is preferably usable as dental root canal filling material or as pulp capping material.

11. A dental composition that comprises at least one acrylamide selected from the group consisting of bisacrylamide, polyacrylamide, bis(meth)acrylamide and poly(meth)acrylamide; a polymerizable monomer, at least one amine or an initiator, and an organic or inorganic filler, which has improved hydrolysis stability.

12. A dental composition as in claim 11, wherein said bisacrylamide if present, has the formula 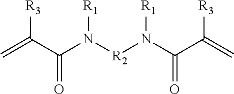
said polyacrylamide if present has the formula 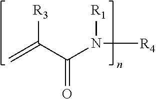
said bis(meth)acrylamide if present has the formula 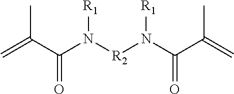
and said poly (meth)acrylamide if present has the formula 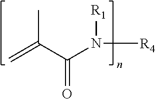
wherein R1 and R3 are the same or different, and are independently H or a substituted or unsubstituted C1 to C18 alkylene, substituted or unsubstituted cycloalkylene, substituted or unsubstituted C5 to C18 arylene or heteroarylene, substituted or unsubstituted C5 to C18 alkylarylene or alkylheteroarylene, substituted or unsubstituted C7 to C30 alkylene arylene; R2 is a difunctional substituted or unsubstituted C1 to C18 alkylene, difunctional substituted or unsubstituted cycloalkylene, difunctional substituted or unsubstituted C5 to C18 arylene or heteroarylene, difunctional substituted or unsubstituted C5 to C18 alkylarylene or alkylheteroarylene, difunctional substituted or unsubstituted C7 to C30 alkylene arylene; and, R4 is a mono- or polyfunctional substituted or unsubstituted C1 to C18 alkylene, mono- or polyfunctional substituted or unsubstituted cycloalkylene, mono- or polyfunctional substituted or unsubstituted C5 to C18 arylene or heteroarylene, mono- or polyfunctional substituted or unsubstituted C5 to C18 alkylarylene or alkylheteroarylene, mono- or polyfunctional substituted or unsubstituted C7 to C30 alkylene arylene.
